(19) United States
(12) Patent Application Publication
(10) Pub. No.: US 2023/0088789 A1
(43) Pub. Date: Mar. 23, 2023

(54) HANDHELD WOUND CARE DEVICE

(71) Applicant: Albert A. Lucio, Winter Haven, FL (US)

(72) Inventor: Albert A. Lucio, Winter Haven, FL (US)

(21) Appl. No.: 17/686,489

(22) Filed: Mar. 4, 2022

Related U.S. Application Data
(60) Provisional application No. 63/246,329, filed on Sep. 21, 2021.

(51) Int. Cl. A61N 5/06

(52) U.S. Cl. CPC A61N 5/0616; A61N 2005/0661; A61N 2005/0659; A61N 2005/0651

(57) ABSTRACT

In some aspects, the techniques described herein relate to a wound care device, including: an ultraviolet (UV) light configured to emit UV light and an infrared (IR) light configured to emit IR light. A method is also disclosed.

### RELATED APPLICATIONS

This application claims the benefit of U.S. Provisional Application No. 63/246,329, filed Sep. 21, 2021, the entirety of which is herein incorporated by reference.

### BACKGROUND

This disclosure relates to a handheld wound care device.

### SUMMARY

In some aspects, the techniques described herein relate to a wound care device, including: an ultraviolet (UV) light configured to emit UV light and an infrared (IR) light configured to emit IR light.

In some aspects, the techniques described herein relate to a device, wherein the UV light emits UV-C light.

In some aspects, the techniques described herein relate to a device, wherein the UV light emits UV light at a wavelength between about 200 and 285 nanometers (nm).

In some aspects, the techniques described herein relate to a device, wherein the UV light emits UV light at a wavelength of about 200 nanometers (nm).

In some aspects, the techniques described herein relate to a device, wherein the IR light emits IR light at a wavelength between about 700 and 900 nanometers (nm).

In some aspects, the techniques described herein relate to a device, wherein the UV light and IR light are arranged in a head adjacent an end of a handle of the device.

In some aspects, the techniques described herein relate to a device, wherein the head is configured to rotate relative to the handle.

In some aspects, the techniques described herein relate to a device, wherein the head is configured to rotate about an axis substantially normal to a length of the handle.

In some aspects, the techniques described herein relate to a device, wherein the head is configured to rotate at a rate of between about 5 to 6 rpm.

In some aspects, the techniques described herein relate to a device, wherein: the device includes a motor, and the device includes a controller configured to command the motor to rotate the head.

In some aspects, the techniques described herein relate to a device, wherein: the device includes an input arranged on the handle, and the device includes a controller configured to command the UV light and IR light to activate in response to a signal from the input.

In some aspects, the techniques described herein relate to a device, where the device includes a battery to power the UV light and the IR light.

In some aspects, the techniques described herein relate to a device, wherein the handle is mountable in a base for charging the battery.

In some aspects, the techniques described herein relate to a device, wherein the device is cordless and handheld.

In some aspects, the techniques described herein relate to a device, wherein a controller is configured to activate the UV and IR light for a period of time.

In some aspects, the techniques described herein relate to a device, wherein the period of time is between 0 and 120 seconds.

In some aspects, the techniques described herein relate to a device, wherein the device is configured such that all operating parameters are pre-set and a user is not allowed to change any operating parameters.

In some aspects, the techniques described herein relate to a method of healing a dermatological wound, including: positioning an ultraviolet (UV) LED of a handheld device near a dermatological wound, the ultraviolet LED configured to emit UV light; and activating the UV LED for a period of time.

In some aspects, the techniques described herein relate to a method, wherein the positioning and activating steps are performed daily for between 7 to 14 consecutive days.

In some aspects, the techniques described herein relate to a method, wherein the device is configured such that a user is not allowed to change the period of time.

### BRIEF DESCRIPTION OF THE DRAWINGS

FIG. 1 illustrates an example wound care device.

FIG. 2 illustrates another view of the example wound care device.

FIG. 3A illustrates a side view of the example wound care device.

FIG. 3B illustrates a side view of the example wound care device without the base.

FIG. 4A illustrates a back view of an example wound care device.

FIG. 4B illustrates a back view of another example wound care device.

FIG. 5 illustrates another view of the example wound care device.

### DETAILED DESCRIPTION

This disclosure relates to a handheld wound care device that utilizes ultraviolet (UV) and/or infrared (IR) light to help heal wounds.

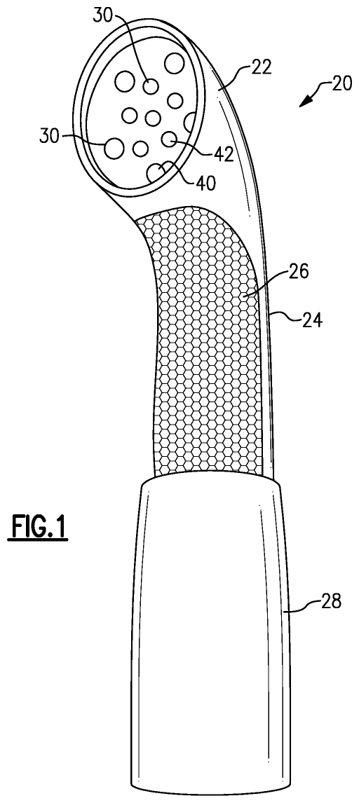

FIG. 1 illustrates an example device 20. The device 20 generally has a head 22 and a handle 24. In some examples, the handle 24 may include a grip portion 26. The grip portion 26 may be textured, for example, in locations where a user's fingers may be placed on the handle 24. In some examples, the device 20 may be placed in a base 28. The head 22 includes a plurality of light emitting diodes (LEDs) 30.

The LEDs 30 may include one or both of UV LEDs 40 and IR LEDs 42. In a particular example, the UV LEDs 40 are configured to emit UV-C light, which is a subtype of UV light especially suited for disinfection. UV-C is relatively short-wavelength UV light, which is known to kill or inactivate microorganisms such as bacteria. The UV LEDs 40 may further emit far-UV-C light. This is light with a longer wavelength than most UV-C light. For example, the UV LEDs 40 may emit light having a wavelength between about 200 and about 285 nm. In another example, the UV LEDs 40 may emit light having a wavelength between about 207 and 222 nm. In a further example, the UV LEDs 40 may emit light with a wavelength of about 222 nm. In another embodiment, the UV LEDs 40 may emit light with a wavelength of about 200 nm.

Far UV-C light may kill bacteria without damaging healthy human cells, such as skin cells. Far UV-C light may also kill fungi. Far UV-C light has the range to traverse microbes that are much smaller in size than human cells, without damaging the human cells. For example, a typical human cell may have a diameter between about 10 and 25 μm, while microbes may have a diameter of less than 1 μm. This may allow for minimal side effects of using the device 20. The IR LEDs 42 may emit light having a wavelength between about 700 and about 900 nm. The IR LEDs 42 may help in collagen reproduction, for example. In the example of FIG. 1, the device 20 includes a combination of UV-C and IR LEDs 40, 42 in the device 20 helps heal wounds on a patient's skin, which may be known as dermatological wounds. The device 20 is not limited for use with humans and could be used to treat wounds of animals, as an example.

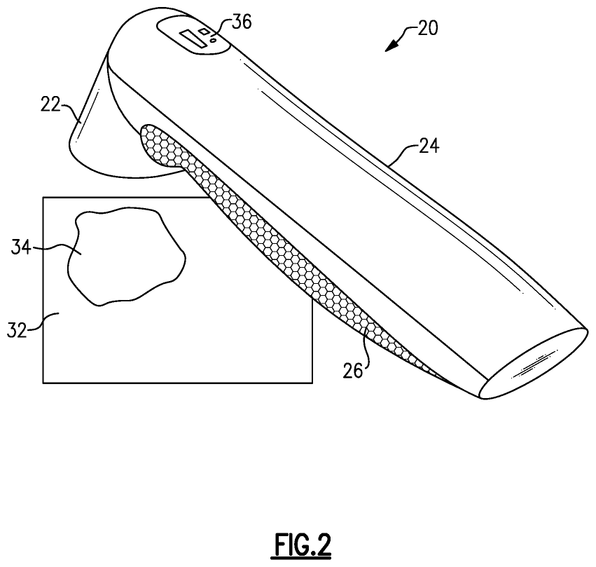

Referring to FIG. 2, and with continuing reference to FIG. 1, the device 20 is placed adjacent a patient's skin 32 in use. In particular, the device 20 is placed near a wound 34 on a patient's skin 32. The device 20 may be placed about 1 inch from the wound 34, for example. The wound 34 may be a cut, scrape, burn, incision, bacterial infection, fungal infection, ulcer, or other skin wound, for example. The device 20 is powered on via an input 36, which may be a button or switch. In this example, the input 36 is on the handle 24. In other examples, the input 36 may be located elsewhere, such as on the head 22 or the base 28. In some examples, the head 22 may rotate relative to the handle 24. In one example, the head 22 rotates at a rate of 5-6 RPM about an axis extending substantially normal to a length of the handle 24.

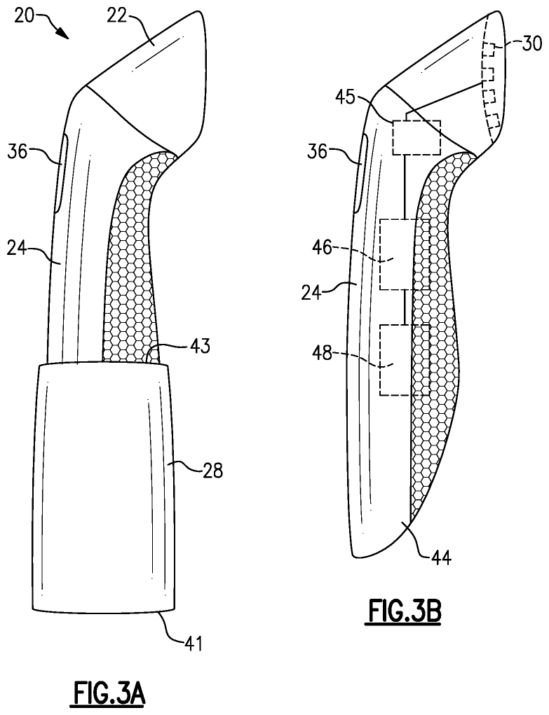

With reference to FIGS. 3A and 3B, the handle 24 removably mounts to the base 28. The base 28 includes a flat bottom 41 for resting on a surface, such as a tabletop. The base 28 is generally cylindrical in shape, with an opening 43 opposite the bottom 41. An end portion 44 of the handle 24 is received within the opening 43, such that the base 28 holds the device 20 upright. The device 20 is removable from the base 28. In one example, the device 20 is a cordless device, and the base 28 recharges batteries within the device 20.

As shown schematically in FIG. 3B, a controller 46 is arranged within the device 20. The controller 46 receives power from one or more batteries, such as battery 48. The battery 48 may be rechargeable, such as through the base 28, or may be a replaceable battery that is not recharged. The controller 46 receives a signal from the input 36, and activates the LEDs 30 in response to the signal. The controller 46 may also begin rotating the head 22 in response to the signal. A motor 45 may be arranged in the device 20 for rotating the head 22. The motor 45 may be in communication with the controller 46.

The device 20 may include a number of customizable operating parameters, which may be set by the user. Customizable parameters may be particularly useful in clinical settings, where users are trained to set these parameters. In a customizable device, the user may be able to set a period of time (i.e., a run time or cycle time) and/or light intensity, as examples. This disclosure also relates to a device which does not allow a user to set any operating parameters, and instead has all parameters pre-set. The latter device may be more user friendly from the perspective of some users, and may be more applicable for residential or over-the-counter applications.

The controller 46 may be programmed with executable instructions for interfacing with and operating the various components of the device 20, including but not limited to those shown in the figures and discussed herein. It should also be understood that the controller 46 may additionally include a combination of hardware and software, and specifically may include a processing unit and non-transitory memory for executing the various control strategies and modes of the device 20. The LEDs 30 are selectively activated in response to instructions from the controller 46.

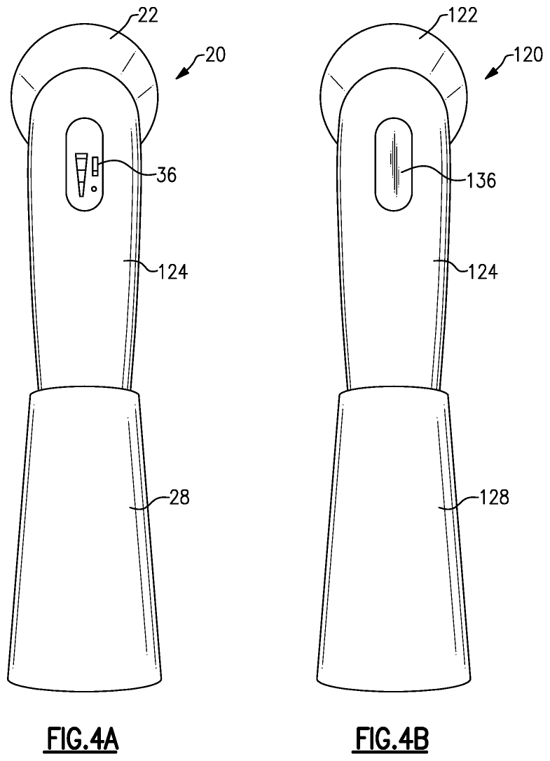

FIG. 4A shows a back view of the device 20. In this example, the input 36 may display information and allow a user to change settings. For example, a user may be able to change a duration that the LEDs are activated (i.e., run time).

FIG. 4B shows a back view of another example device 120. To the extent not otherwise described or shown, the device 120 in FIG. 4B corresponds to the device 20 of FIGS. 1-4A, with like parts having reference numerals preappended with a “1.” The device 120 includes an input 136 that does not display information or provide the ability to change settings. For example, the device 120 may be pre-programmed to run a treatment cycle when a user depresses the input 136. The device 120 may be pre-programmed to run for a set time, which is a predefined period of time. In one example, the predefined period of time is between about 1 and about 10 minutes. In a further embodiment, the predefined period of time is between about 30 and 120 seconds. In a further embodiment, the predefined period of time is about 60 seconds. In another embodiment, the predefined period of time is about 120 seconds. In some examples, the device 120 may be used daily. The device 120 may be used daily for 7 to 14 days, in one example. In other examples, the device 120 may be used more or less frequently. For example, the device 120 may be used 3 times per week. The device 120 may not require the user to set any operating parameters of the treatment cycle, and instead merely requires the user to place the device 120 near a wound, and press the “on” button. Such a pre-programmed device 120 may be particularly useful in residential or over-the-counter applications.

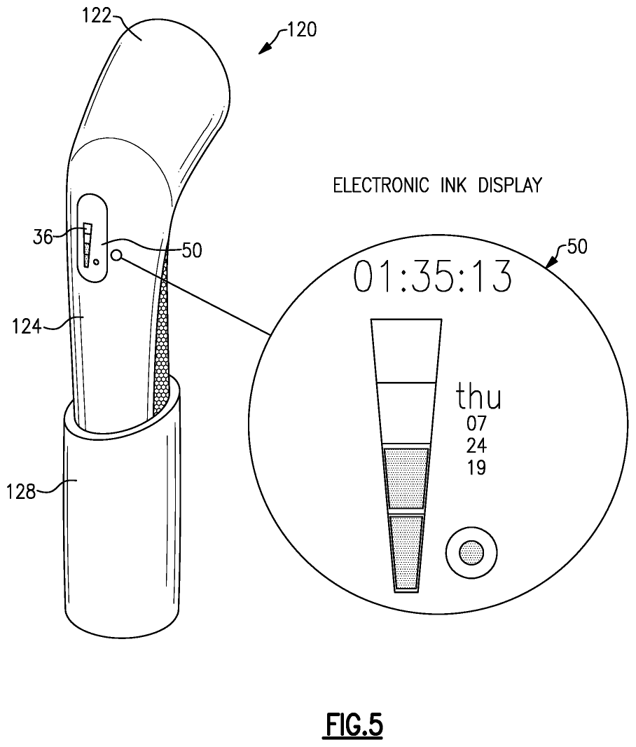

FIG. 5 illustrates a view of the example wound care device 20 of FIG. 4A. In this example, the device 20 includes a display 50. The display 50 may be arranged on the input 36, or may be arranged elsewhere on the device 120. The display 50 may be an electronic ink display, for example. The display 50 may display information to a patient or user. The display 50 may include information such as a date, time, time the device has been on, time remaining in treatment, power indicator, and/or battery life.

The devices 20, 120 provide a reliable, quick, and relatively easy solution for disinfecting wounds. The combination of UV-C and IR light helps kill bacteria and rebuild collagen without damaging the skin. The ease of use of the devices 20, 120, coupled with the relatively fast process, relieves a burden on patients. The devices 20, 120 are also less expensive than other higher cost systems on the market. The use of UV-C and IR light may also help kill bacteria that are resistant to antibiotics.

It should be understood that terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

1. A wound care device, comprising:
an ultraviolet (UV) light configured to emit UV light and an infrared (IR) light configured to emit IR light.

2. The device as recited in claim 1, wherein the UV light emits UV-C light.

3. The device as recited in claim 2, wherein the UV light emits UV light at a wavelength between about 200 and 285 nanometers (nm).

4. The device as recited in claim 2, wherein the UV light emits UV light at a wavelength of about 200 nanometers (nm).

5. The device as recited in claim 1, wherein the IR light emits IR light at a wavelength between about 700 and 900 nanometers (nm).

6. The device as recited in claim 1, wherein the UV light and IR light are arranged in a head adjacent an end of a handle of the device.

7. The device as recited in claim 6, wherein the head is configured to rotate relative to the handle.

8. The device as recited in claim 7, wherein the head is configured to rotate about an axis substantially normal to a length of the handle.

9. The device as recited in claim 8, wherein the head is configured to rotate at a rate of between about 5 to 6 rpm.

10. The device as recited in claim 8, wherein:
the device includes a motor, and the device includes a controller configured to command the motor to rotate the head.

11. The device as recited in claim 6, wherein:
the device includes an input arranged on the handle, and the device includes a controller configured to command the UV light and IR light to activate in response to a signal from the input.

12. The device as recited in claim 1, where the device includes a battery to power the UV light and the IR light.

13. The device as recited in claim 12, wherein the handle is mountable in a base for charging the battery.

14. The device as recited in claim 1, wherein the device is cordless and handheld.

15. The device as recited in claim 1, wherein a controller is configured to activate the UV and IR light for a period of time.

16. The device as recited in claim 15, wherein the period of time is between 0 and 120 seconds.

17. The device as recited in claim 1, wherein the device is configured such that all operating parameters are pre-set and a user is not allowed to change any operating parameters.

18. A method of healing a dermatological wound, comprising:
positioning an ultraviolet (UV) LED of a handheld device near a dermatological wound, the ultraviolet LED configured to emit UV light; and
activating the UV LED for a period of time.

19. The method as recited in claim 18, wherein the positioning and activating steps are performed daily for between 7 to 14 consecutive days.

20. The method as recited in claim 18, wherein the device is configured such that a user is not allowed to change the period of time.
